(19) United States
(12) Patent Application Publication
(10) Pub. No.: US 2023/0066648 A1
(43) Pub. Date: Mar. 2, 2023

(54) METHOD FOR INDUCING TRANSDIFFERENTIATION OF SOMATIC CELLS INTO MAMMARY EPITHELIAL CELLS IN VITRO USING SMALL MOLECULE COMPOUND

(71) Applicants: GUANGXI UNIVERSITY, Nanning (CN); Guangxi Benpeng Biotechnology Co., Ltd., Nanning (CN)

(72) Inventors: Ben HUANG, Nanning (CN); Dandan ZHANG, Nanning (CN); Sheng YE, Nanning (CN); Shaoqian ZHU, Nanning (CN); Quanhui LIU, Nanning (CN); Liangshan QIN, Nanning (CN); Deshun SHI, Nanning (CN); Jigang HU, Nanning (CN); Xiaolian XIE, Nanning (CN)

(73) Assignees: GUANGXI UNIVERSITY, Nanning (CN); Guangxi Benpeng Biotechnology Co., Ltd., Nanning (CN)

(21) Appl. No.: 17/795,607

(22) PCT Filed: Jun. 30, 2020
(86) PCT No.: PCT/CN2020/099117

§ 371 Date: Jul. 27, 2022

(30) Foreign Application Priority Data
Dec. 20, 2019 (CN) 201911328267.2

(51) Int. Cl. C12N 5/071

(52) U.S. Cl. CPC C12N 5/0631; C12N 2500/44; C12N 2501/999

(57) ABSTRACT

The present disclosure provides a method for inducing transdifferentiation of somatic cells into mammary epithelial cells in vitro using a small molecule compound. The method includes: inhibiting an expression of TGFbeta R1 and related sites thereof, to induce the transdifferentiation of the somatic cells into the mammary epithelial cells in vitro. The present disclosure fills a gap in the technology of inducing the transdifferentiation of fibroblasts to the mammary epithelial cells using the small molecule compound; the present disclosure also provides a research platform for in vitro researches on a mammary gland bioreactor, mammary gland development and differentiation, breast cancer, and transdifferentiation of the fibroblasts into other types of functional cells.

### TECHNICAL FIELD

The present disclosure belongs to the technical field of cell transdifferentiation, and in particular relates to a method for inducing transdifferentiation of somatic cells into mammary epithelial cells in vitro using a small molecule compound.

### BACKGROUND ART

Mammary epithelial cells are an in vitro model used for studying mammary gland growth and development and lactation mechanism, and verifying the effectiveness of breast tissue-specific expression vectors. At present, most primary mammary epithelial cells are cultured by collagenase digestion and tissue block culture.

Mammary gland tissues are digested with collagenase and then subjected to density gradient centrifugation to obtain relatively-pure epithelial cells. The tissue block culture is simple to operate, saves tissue samples, and avoids the adverse effects of digestion and centrifugation on cells. However, it takes a long time for the cells to grow from the tissue block. Connective tissue cells such as fibroblasts grow first, and epithelial cells appear more slowly in a large amount. Culture of primary mammary epithelial cells yields a mixture of epithelial cells and fibroblasts, whether by the collagenase digestion or the tissue block culture.

However, no matter whether the collagenase digestion or the tissue block culture is used, there are problems of limited in vitro proliferative capacity of mammary epithelial cells and lack of lactation function.

In recent years, the combination of small molecule compounds has been used in human, mouse and other species to achieve the transformation of various cells such as nerve cells, cardiomyocytes, pancreatic cells, and liver cells. However, no method has been reported to induce transdifferentiation of terminally-differentiated somatic cells into mammary epithelial cells in any species.

### SUMMARY

In view of this, the present disclosure aims to propose a method for inducing transdifferentiation of somatic cells into mammary epithelial cells in vitro using a small molecule compound. The present disclosure fills a gap in the technology of inducing fibroblasts to transform into mammary epithelial cells using the small molecule compound, and can continuously obtain low-generation and functional mammary epithelial cells by induction of the somatic cells.

To achieve the above objective, the present disclosure adopts the following technical solutions.

The present disclosure provides a method for inducing transdifferentiation of somatic cells into mammary epithelial cells in vitro, including: inhibiting an expression of TGFbeta R1 and related sites thereof.

Preferably, the method may include: inhibiting the TGFbeta R1 and the related sites thereof using a small molecule compound or gene interference, where the small molecule compound includes one or more of valproic acid (VPA), Forskolin, Tranylcypromine, TTNPB (Arotinoid Acid), RepSox, SB431542, SB525334, and LDN193189.

The present disclosure further provides an induction medium for inducing transdifferentiation of somatic cells into mammary epithelial cells in vitro, including a basal solution, KnockOut Serum Replacement (KSR), a non-essential amino acid, β-mercaptoethanol, and a small molecule compound; where preferably, the small molecule compound includes one or more of VPA, Forskolin, Tranylcypromine, TTNPB, RepSox, SB431542, SB525334, and LDN193189; in a final medium, the VPA, the Forskolin, the Tranylcypromine, the TTNPB, the RepSox, the SB431542, the SB525334, and the LDN193189 have concentrations of 0 mM to 4 mM, 0 μM to 80 μM, 0 μM to 80 μM, 0 μM to 8 μM, 0 μM to 80 μM, 0 μM to 80 μM, 0 μM to 80 μM, and 0 μM to 80 μM, respectively; and the concentration of each above substance is not 0 simultaneously.

Preferably, the small molecule compound may include the VPA, the Forskolin, the Tranylcypromine, the TTNPB, and the Repsox, with concentrations of 0.0625 mM to 4 mM, 1.25 μM to 80 μM, 1.25 μM to 80 μM, 0.125 μM to 8 μM, and 1.25 μM to 80 μM in the final medium, respectively, preferably 0.25 mM to 2 mM, 5 μM to 40 μM, 5 μM to 40 μM, 0.5 μM to 4 μM, and 5 μM to 40 μM, respectively; more preferably, the Repsox may be replaced by one of the SB431542, the SB525334, and the LDN193189.

Preferably, the basal solution, the KSR, the non-essential amino acid, and the β-mercaptoethanol may have a volume ratio of 78:20:1:1; more preferably, the base solution may be N2B27, including Knockout Dulbecco's Modified Eagle Medium: F-12 (DMEM/F12), N-2 Supplement (N2, 100×), Neurobasal, B-27 Supplement (B27, 50×), Glutamine (100×), with a volume ratio of 99:1:97:2:1.

The present disclosure further provides use of the induction medium in inducing transdifferentiation of somatic cells into mammary epithelial cells in vitro.

The present disclosure further provides a method for inducing transdifferentiation of somatic cells into mammary epithelial cells in vitro by using an induction medium, including the following steps:

1) inoculating the somatic cells into a petri dish; adding a high-glucose dulbecco's modified eagle medium and 10% fetal bovine serum medium (DMEM+10% FBS), and placing in an incubator at 37° C. and a humidity of 95% with 5% carbon dioxide; and
2) after conducting culture for 8 h to 24 h, replacing the induction medium according to any one of claims 3 to 5; continuing induction culture for 8 d, where a new induction medium is replaced every two days; obtaining transdifferentiated mammary epithelial cells.

Preferably, the method may further include step 3): digesting the transdifferentiated mammary epithelial cells obtained in step 2) with trypsin and conducting subculture onto a culture plate pretreated with a Matrix substrate; replacing a mammary epithelial medium to continue culture, followed by conducting subculture or cryopreservation; where Matrix and gelatin in the Matrix substrate have a volume percentage of 1:(50-100).

Preferably, the somatic cells may be ear fibroblasts or epidermal cells derived from a human being, a mouse, a rat, a rabbit, a pig, a sheep, a goat, a bovine, or a buffalo. Compared with the prior art, the method for inducing transdifferentiation of somatic cells into mammary epithelial cells in vitro using a small molecule compound has the following advantages:

(1) The present disclosure fills a gap in the technology of inducing the transdifferentiation of fibroblasts to the mammary epithelial cells using the small molecule compound. Since no method has been reported to induce transdifferentiation of terminally-differentiated somatic cells into mammary epithelial cells in any species.

(2) The present disclosure provides a research platform for in vitro researches on mammary gland development and differentiation and breast cancer.

(3) The present disclosure provides a research platform for in vitro researches on the transdifferentiation of fibroblasts into other types of functional cells.

(4) The present disclosure provides a new method for the production of transgenic mammary gland-based bioreactors. An exogenous gene of a medicinal protein can be overexpressed in the somatic cells and then subjected to induction, and induced mammary epithelial cells can express the medicinal protein, which is faster than obtaining transgenic animals to produce the medicinal protein.

(5) The present disclosure can also avoid the problems of limited in vitro proliferative capacity of mammary epithelial cells and lack of lactation function. Since fibroblasts of an individual are ubiquitous. Even if mammary epithelial cells have no function or lose an ability to proliferate in vitro, induction can be conducted by the fibroblasts to continuously obtain low-passage and functional mammary epithelial cells.

### BRIEF DESCRIPTION OF THE DRAWINGS

FIG. 1 shows a time pathway of transdifferentiation of fibroblasts into mammary epithelial cells induced by a small molecule compound;

FIG. 2 shows a morphological change process of transdifferentiation of fibroblasts into mammary epithelial cells induced by the small molecule compound;

FIG. 3 shows that transdifferentiated goat mammary epithelial cells (CiMECs, left side) and milk-separated and cultured goat mammary epithelial cells (GMECs, right side) have similar cell morphological characteristics;

FIG. 4 shows results of immunofluorescence showing that mammary epithelial cells (CiMECs) obtained by BFRTV-induced (include TTNPB(B), Forskolin(F), Repsox(R), Tranylcypromine(T), valproic acid (V)) fibroblast transdifferentiation express mammary epithelial cell-specific antigens E-cadherin, cytokeratin 8 (KRT8), cytokeratin 18 (KRT18), integrin-α6 (CD49f), Epithelial cell adhesion molecule (EpCAM), and SRY (sex determining region Y)-box 9 (SOX9);

FIG. 5 shows results of quantitative polymerase chain reaction (PCR) showing that mammary epithelial cells (CiMECs) obtained by BFRTV-induced fibroblast transdifferentiation significantly express mammary epithelial cell marker genes; and the expression of fibroblast marker genes is significantly down-regulated;

FIG. 6 shows western bolt (WB) results showing that mammary epithelial cells (CiMECs) obtained by BFRTV-induced fibroblast transdifferentiation express beta-casein (CSN2) and lactoferrin (LTF);

FIG. 7 shows a cell morphology of CiMECs obtained from fibroblasts induced by overall adjustment of BFRTV concentration;

FIG. 8 shows a morphological diagram of transdifferentiation of fibroblasts into mammary epithelial cells under different concentrations of Repsox (R induction medium) alone;

FIG. 9 shows a cell morphology of fibroblasts induced by other inhibitors (SB431542 (4), SB525334 (5), and LDN193189 (L)) for 8 d; and regardless of the combination of small molecule compounds (BFTV4/BFTV5/BFTVL) or the use of small molecule compounds SB431542 (4), SB525334 (5), LDN193189 (L) alone, the fibroblasts can be induced into mammary epithelial cells; and

FIG. 10 shows that interference with Transforming Growth Factor Beta Receptor I (TGFbeta R1) expression has induced the fibroblasts for eight days to form a cell morphology similar to that of BFRTV induction.

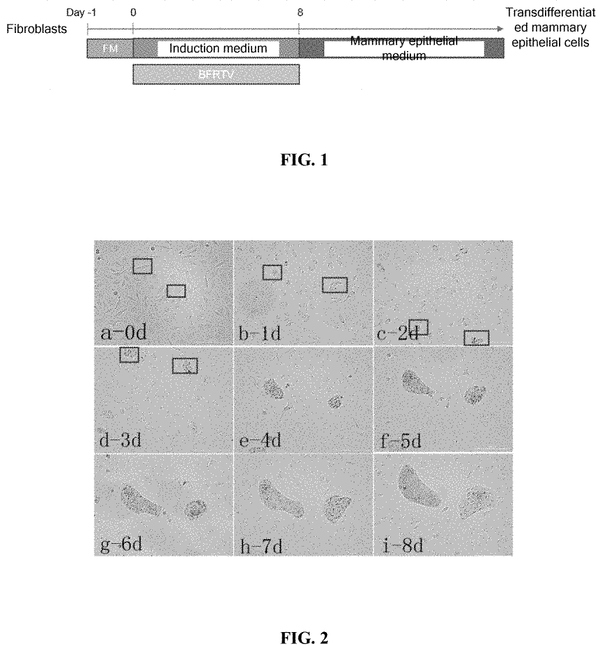

### DETAILED DESCRIPTION OF THE EMBODIMENTS

Unless otherwise defined, the technical and scientific terms used in the following examples have the same meanings as commonly understood by those skilled in the art to which the present disclosure belongs. These examples are disclosed herein and based on the general level of those skilled in the art, those skilled in the art will understand that the following are for illustration only and that various changes, modifications and adaptations may be made without departing from the scope of the present disclosure. Unless otherwise specified, in the following examples, the test reagents used are all conventional biochemical reagents, and the test methods are all conventional methods. The techniques involved, unless otherwise specified, are conventional techniques in various fields such as molecular biology, cell biology, and biochemistry well-known to those skilled in the art.

The present disclosure will be described in detail below with reference to the accompanying drawings and the examples.

A medium involved in the following examples is as follows:

1. Induction medium (BFRTV) components:

a basal solution (N2B27): a 200 mL system:

| | |
|---|---|
| Knockout DMEM/F12 | 99 mL |
| N2 (100×) | 1 mL |
| Neurobasal | 97 mL |
| B27 (50×) | 2 mL |
| Glutamine (100×) | 1 mL |

Induction medium (BFRTV) of a 100 mL system

| Component | Volume/Consentration |
|---|---|
| N2B27 | 78 mL |
| KSR | 20 mL |
| Non-essential amino acid (100×) | 1 mL |
| β-mercaptoethanol (10×) | 1 mL |
| VPA(V) | 0.5 mM |
| Forskolin(F) | 10 μM |
| Tranylcypromine(T) | 10 μM |
| TTNPB(B) | 1 μM |
| Repsox(R) | 10 μM |

BFTV induction medium is the above BFRTV medium without a small molecule compound R;
R induction medium is N2B27+KSR+non-essential amino acid+β-mercaptoethanol+Repsox (R);
BFTV4 induction medium is 10 μM SB431542 (4) instead of the small molecule compound R in the BFRTV medium;
BFTV5 induction medium is 5 μM SB525334 (5) instead of the small molecule compound R in the BFRTV medium;
BFTVL induction medium is 1 μM LDN193189 (L) instead of the small molecule compound R in the BFRTV medium;
SB431542 (4) induction medium is N2B27+KSR+non-essential amino acids+β-mercaptoethanol+10 μM SB431542 (4);
SB525334(5) induction medium is N2B27+KSR+non-essential amino acids+β-mercaptoethanol+5 μM SB525334 (5);
LDN193189 (L) induction medium is N2B27+KSR+non-essential amino acids+β-mercaptoethanol+1 μM LDN193189 (L).

In the above induction media, a volume ratio of N2B27+KSR+non-essential amino acid+β-mercaptoethanol remains unchanged, and only a concentration of the small molecule compounds is adjusted.

2. Composition of a mammary epithelial medium (with 100 mL as an example) 88.38 mL of DMEM/F12+10 mL of FBS+0.5 mL of hydrocortisone (200×)+0.1 mL of heparin (1000×)+0.01 mL of EGF (Epidermal Growth Factor, 10000×)+0.01 mL of IGF-1 (insulin-like growth factors-1, 10000×)+1 mL penicillin-streptomycin (100×).

### Example 1

A method for inducing transdifferentiation of somatic cells into mammary epithelial cells in vitro using a small molecule compound and an experiment of detection were provided, and specific operations were as follows:

1. The ear margin fibroblasts (GEFs) of goat were isolated and cultured by a tissue adherence method to provide cell materials for subsequent induction.

Goats aged 30 d to 60 d were selected, and the ear margin skin was sterilized, the marginal tissue blocks were cut with a scalpel, and washed 2 to 3 times in a PBS buffer containing double antibodies, stored in a high-glucose DMEM+10% FBS (volume percentage) medium. Tissue block was treated in the laboratory, including alcohol sterilization, removal of hair and cartilage in PBS buffer, and washing with the PBS buffer three times after removal. The treated tissue blocks were placed in a 1.5 mL centrifuge tube, cut off to an appropriate size with ophthalmic scissors, and evenly spread into a 60 mm cell culture dish, placed upside down in an incubator.

When the tissue blocks adhered well, DMEM medium was added for adherent culture, and the medium was replaced every 2 d. When the primarily-cultured monolayer cells reached 80% to 90% confluence in the culture dish, subculture was conducted, the old medium was discarded, digested with 0.25% trypsin (mass percentage), and treated in a high-glucose DMEM+10% FBS (volume percent) medium for neutralization. A cell suspension was collected, centrifuged (1200 r/min, 3 min), and a supernatant was discarded, and the cells were resuspended and inoculated evenly.

2. The fibroblasts were inoculated into a 60 mm cell culture dish at a density of 5×10⁵, and after culturing for 8 h to 24 h in the high-glucose DMEM+10% FBS (volume percentage), the induction medium BFRTV was replaced, and then cultured at 37° C. for 8 d in a 5% CO₂ incubator, where the medium was changed every two days. The morphological changes of cells during the induction were shown in FIG. 2, and the induced transdifferentiated mammary epithelial cells (CiMECs) were obtained after 8 d of induction.

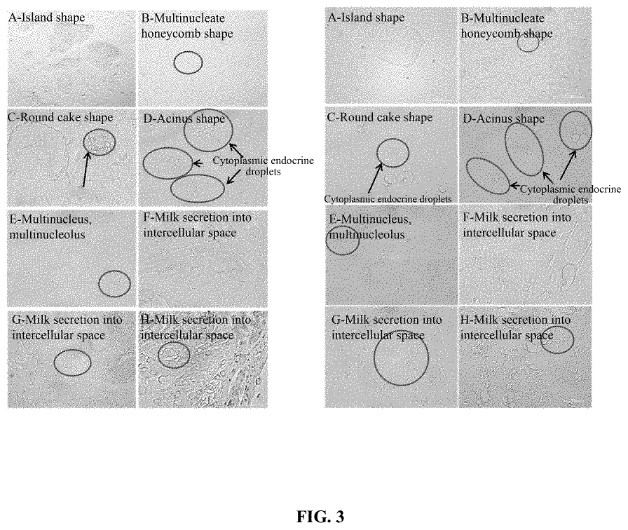

3. When cultured to the 8th day, CiMECs can be subcultured and inoculated onto a plate pretreated with Matrix (a volume ratio of Matrix to gelatin was 1:50), and then cultured in a mammary epithelial cell medium, when the cell confluence reached about 90%, the cells were subcultured or frozen for subsequent detection. The cell morphology of CiMECs after 8 d of induction and subculture was similar to that of goat mammary epithelial cells (GMECs) (FIG. 3).

4. The specific antigens of mammary epithelial cells were detected on cells induced by BFRTV induction medium for 4 d (BFRTV-4d), 8 d (BFRTV-8d), and transdifferentiated mammary epithelial cells (CiMECs).

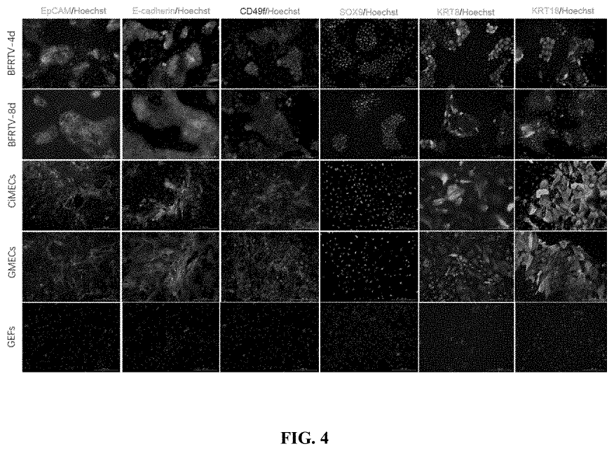

The method included the following specific steps: The cells of BFRTV-4d, BFRTV-8d and CiMECs in the culture plate were fixated with 4% paraformaldehyde (PFA) at room temperature for 30 min; the cells were washed with a blocking solution three times, 5 min in each time; the cells were permeabilized by 1% Triton X-100 (volume percentage) for 15 min at room temperature; the cells were washed with the blocking solution three times again; non-specific sites were blocked with 5% donkey serum, and then blocked for 2 h at room temperature; the cells were washed three times with TBP (Tritonx-Bovine albumin-Phosphate Buffer Saline) for 5 min in each time; a primary antibody was added for incubation at 4° C. overnight; on a next day, the culture plate was placed at room temperature, rewarmed for 20 min, and then washed with TBP three times for 5 min in each time, a secondary antibody and a Hoechst mixture were added in the dark, and incubated at room temperature for 1 h; after washing three times with a TBP solution, fluorescence microscope observation and photographing experiment were conducted. The results of immunofluorescence staining (FIG. 4) showed that BFRTV-4d, BFRTV-8d, CiMECs and GMECs all expressed mammary epithelial cell marker antigens, including E-cadherin, cytokeratin 8 (KRT8), cytokeratin 18 (KRT18), integrin-α6 (CD49f), EpCAM and SOX9, but not in goat ear fibroblasts (GEFs).

5. Detection of expression of mammary epithelial cell marker genes by quantitative PCR (qPCR).

The specific operation steps were as follows: (1) total RNA extraction: The medium was discarded, cells were washed three times with PBS, and lysed on ice for 5 min with 1 ml of pre-cooled TRIZOL; 200 μL of chloroform was added, shaken vigorously for 15 sec, and placed on ice for 5 min; the cells were centrifuge at 12000 r/min for 15 min at 4° C.; an upper aqueous phase was transferred to pre-cooled isopropanol, inverted and mixed, and placed on ice for 5 min; the cells were centrifuged at 12000 r/min for 10 min at 4° C.; a supernatant was discarded, 1 mL of pre-chilled 75% ethanol (volume percentage) was added, RNA was suspended by flicking a bottom of the tube with fingertips, the RNA and the tube wall were washed thoroughly, and centrifuged at 7500 r/min at 4° C. for 8 min; a supernatant was discarded, when the precipitate was translucent, an appropriate amount of diethypyrocarbonate (DEPC) treated water was added to completely dissolve the RNA, 1 μL of the RNA was collected for purity and integrity testing, and the rest was reverse-transcribed or frozen in a −80° C. refrigerator. (2) cDNA template preparation. A Vazyme R223-01 synthesis kit was used according to the instructions. 3. Fluorescence quantification PCR. A Vazyme Q711-02/03 reagent was used according to the instructions. The results of qPCR (FIG. 5) showed that compared with BFRTV-Od, the BFRTV-4d, BFRTV-8d, CiMECs and GMECs highly expressed mammary epithelial cell-related marker genes CDH1, EPCAM, KRT19, ITGA6, INSR, PRLR, ELF5, and LTF, while the expression levels of fibroblast marker genes COL6A2 and FBN1 were significantly down-regulated.

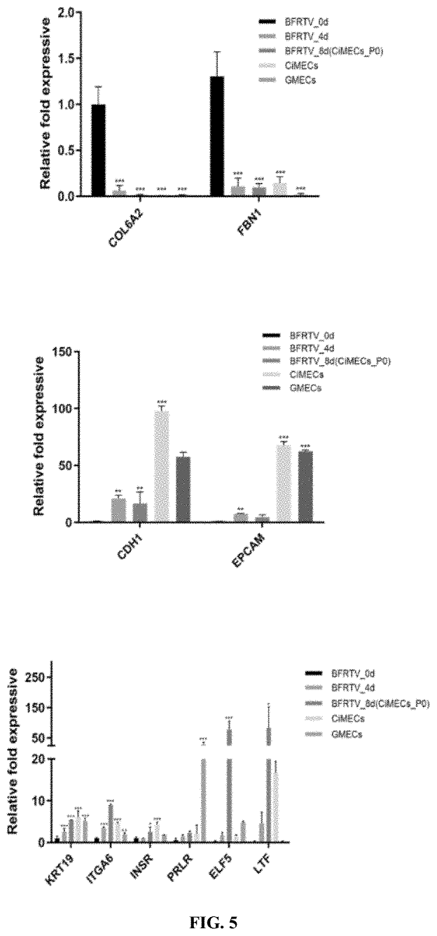

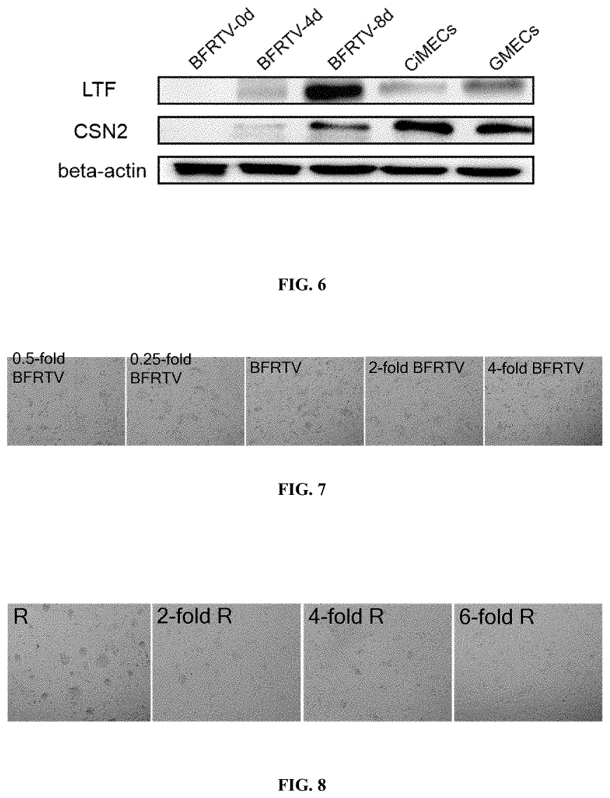

6. WB detection of lactation-related protein expression. The results showed (FIG. 6) that BFRTV-4d, BFRTV-8d, CiMECs and GMECs significantly expressed mammary epithelial cell-specific secretory proteins, including lactoferrin (LTF) and beta casein (CSN2).

The specific operation steps were as follows: cells were lysed in a denaturing lysis buffer containing a protease inhibitor for 30 min, centrifuged at 12,000 rpm/min, and 4° C. for 10 min; a protein concentration in the lysate was determined with a Bicinchoninic Acid Assay (BCA) protein detection kit; the proteins were separated by sodium dodecyl sulfate polyacrylamide gel electrophoresis (SDS-PAGE) with a 12% protein gel (mass percentage), and then the proteins were transferred to a nitrocellulose filter, and then blocked by non-fat milk powder at room temperature for 1 h; a primary antibody was incubated overnight at 4° C.; on a next day, a secondary antibody was incubated at room temperature for 1 h; development was conducted.

In summary, through the morphological observation and comparison, the detection of marker genes and specific antigens, and the identification of lactation ability, it can be proved that the cells obtained by transdifferentiation induced by five small molecule compounds (BFRTV) are mammary epithelial cells with lactation function.

### Example 2

Taking the ear margin fibroblasts of Guanzhong dairy goat as an experimental object, the concentration of five small molecule compounds currently used was adjusted, and the concentration of BFRTV was adjusted as a whole under the condition that the basal solution remained unchanged. The results showed that (shown in FIG. 7), where the rest of the experimental steps and experimental parameters were the same as in Example 1; after induction, the cells still had the BFRTV-like cell morphology, that is, BFRTV concentrations within 0.5 times to 4 times could induce the mammary epithelial cells.

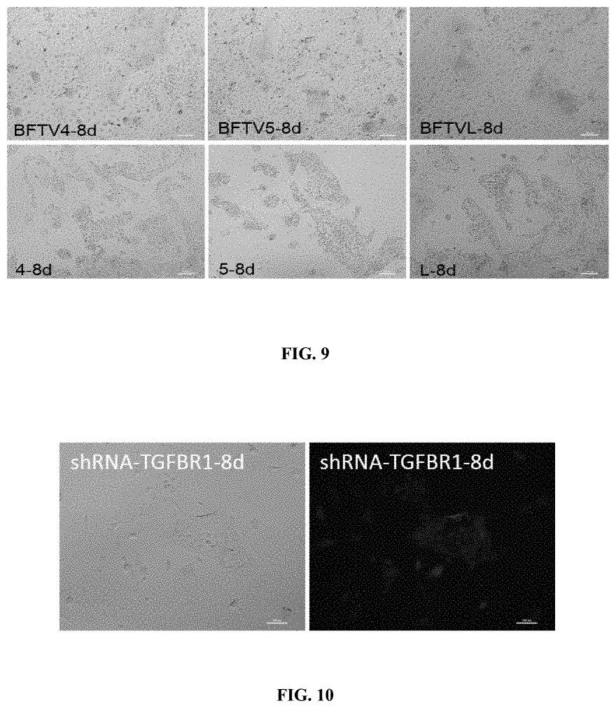

Through screening, it was found that the mammary epithelial cells (CiMECs) similar to those by BFRTV induction medium were obtained by using TGFbeta R1 inhibitor Repsox (R induction medium) alone, and then the concentration of R was screened, and the results showed (FIG. 8) that mammary epithelial cells could be generated in the range of 1 to 8 times the concentration. Meanwhile, TGFbeta R1 and related site inhibitors SB431542 (4), SB525334 (5), and LDN193189 (L) thereof were used to replace the small molecule compound R in BFRTV, to form BFTV4, BFTV5, and BFTVL induction mediums, respectively; fibroblasts could also be transdifferentiated into mammary epithelial cells. In addition, the SB431542 (4), SB525334 (5), and LDN193189 (L) induction mediums alone could still induce the transdifferentiation of fibroblasts into mammary epithelial cells (FIG. 9). This indicated that small molecule compounds that inhibit TGFbeta R1 and its related sites were the key to obtaining transdifferentiated mammary epithelial cells (CiMECs).

### Example 3

Down-regulation of TGFbeta R1 expression on fibroblasts by gene interference technology could also induce transdifferentiation of fibroblasts into mammary epithelial cells.

A lentiviral recombinant plasmid pSicoR-Ef1a-mCh TGFBR1 shRNA was constructed, co-transfected 293T cells with Vesicular stomatitis virus-G (VSVG) and nuclear respiratory factor (NRF) using Lipofectamine™ 3000 for lentiviral packaging, and then infected fibroblasts with the packaged lentivirus. The lentivirus-infected cells were cultured in BFTV induction medium, at 37° C., 95% saturated humidity, in a 5% CO₂ incubator. It was found that mammary epithelial cells similar to those induced by BFRTV medium could be formed after 8 d of culture (FIG. 10). This indicated that inhibiting the expression of TGFbeta R1 was the key to inducing transdifferentiated mammary epithelial cells (CiMECs) in vitro.

The above described are merely preferred embodiments of the present disclosure, and not intended to limit the present disclosure. Any modifications, equivalent replacements and improvements made within the spirit and principle of the present disclosure should all fall within the scope of protection of the present disclosure.

What is claimed is:

1. A method for inducing transdifferentiation of somatic cells into mammary epithelial cells in vitro, comprising: inhibiting an expression of TGFbeta R1 and related sites thereof.

2. The method according to claim 1, comprising: inhibiting the TGFbeta R1 and the related sites thereof using a small molecule compound or gene interference, wherein the small molecule compound comprises one or more of valproic acid (VPA), Forskolin, Tranylcypromine, Arotinoid Acid (TTNPB), RepSox, SB431542, SB525334, and LDN193189.

3. An induction medium for inducing transdifferentiation of somatic cells into mammary epithelial cells in vitro, comprising a basal solution, KnockOut Serum Replacement (KSR), a non-essential amino acid, β-mercaptoethanol, and a small molecule compound; wherein preferably, the small molecule compound comprises one or more of VPA, Forskolin, Tranylcypromine, TTNPB, RepSox, SB431542, SB525334, and LDN193189; in a final medium, the VPA, the Forskolin, the Tranylcypromine, the TTNPB, the RepSox, the SB431542, the SB525334, and the LDN193189 have concentrations of 0 mM to 4 mM, 0 μM to 80 μM, 0 μM to 80 μM, 0 μM to 8 μM, 0 μM to 80 μM, 0 μM to 80 μM, 0 μM to 80 μM, and 0 μM to 80 μM, respectively; and the concentration of each above substance is not 0 simultaneously.

4. The induction medium according to claim 3, wherein the small molecule compound comprises the VPA, the Forskolin, the Tranylcypromine, the TTNPB, and the RepSox, with concentrations of 0.0625 mM to 4 mM, 1.25 μM to 80 μM, 1.25 μM to 80 μM, 0.125 μM to 8 μM, and 1.25 μM to 80 μM in the final medium, respectively, preferably 0.25 mM to 2 mM, 5 μM to 40 μM, 5 μM to 40 μM, 0.5 μM to 4 μM, and 5 μM to 40 μM, respectively; more preferably, the Repsox is replaced by one of the SB431542, the SB525334, and the LDN193189.

5. The induction medium according to claim 3, wherein the basal solution, the KSR, the non-essential amino acid, and the β-mercaptoethanol have a volume ratio of 78:20:1:1; more preferably, the base solution is N2B27, comprising Knockout Dulbecco's Modified Eagle Medium: F-12 (DMEM/F12), N-2 Supplement (N2, 100×), Neurobasal, B-27 Supplement (B27, 50×), Glutamine (100×), with a volume ratio of 99:1:97:2:1.

6. Use of the induction medium according to claim 3 in inducing transdifferentiation of somatic cells into mammary epithelial cells in vitro.

7. A method for inducing transdifferentiation of somatic cells into mammary epithelial cells in vitro by using an induction medium, comprising the following steps:
1) inoculating the somatic cells into a petri dish; adding a high-glucose dulbecco's modified eagle medium and 10% fetal bovine serum medium (DMEM+10% FBS), and placing in an incubator at 37° C. and a humidity of 95% with 5% carbon dioxide; and
2) after conducting culture for 8 h to 24 h, replacing the induction medium according to claim 3; continuing induction culture for 8 d, wherein a new induction medium is replaced every two days; obtaining transdifferentiated mammary epithelial cells.

8. The method according to claim 7, further comprising step 3): digesting the transdifferentiated mammary epithelial cells obtained in step 2) with trypsin and conducting subculture onto a culture plate pretreated with a Matrix substrate; replacing a mammary epithelial medium to continue culture, followed by conducting subculture or cryopreservation; wherein Matrix and gelatin in the Matrix substrate have a volume percentage of 1:(50-100).

9. The method according to claim 1, wherein the somatic cells are ear fibroblasts or epidermal cells derived from a human being, a mouse, a rat, a rabbit, a pig, a sheep, a goat, a bovine, or a buffalo.

10. The induction medium according to claim 3, wherein the somatic cells are ear fibroblasts or epidermal cells derived from a human being, a mouse, a rat, a rabbit, a pig, a sheep, a goat, a bovine, or a buffalo.

11. The use according to claim 6, wherein the somatic cells are ear fibroblasts or epidermal cells derived from a human being, a mouse, a rat, a rabbit, a pig, a sheep, a goat, a bovine, or a buffalo.

12. The method according to claim 7, wherein the somatic cells are ear fibroblasts or epidermal cells derived from a human being, a mouse, a rat, a rabbit, a pig, a sheep, a goat, a bovine, or a buffalo.
